(19) United States
(12) Patent Application Publication
(10) Pub. No.: US 2017/0007762 A1
(43) Pub. Date: Jan. 12, 2017

(54) INFUSION OF INSULIN INTO A PATIENT AND DIABETES MELLITUS MEDICAL METHODS BASED ON THE PATIENTS MONITORED ANALYTE CONCENTRATION

(71) Applicant: ABBOTT DIABETES CARE INC., Alameda, CA (US)

(72) Inventors: Gary A. Hayter, Oakland, CA (US); John C. Mazza, Long Beach, CA (US); Jai Karan, Walnut, CA (US); Saeed Nekoomaram, San Mateo, CA (US)

(21) Appl. No.: 15/274,694

(22) Filed: Sep. 23, 2016

Related U.S. Application Data
(62) Division of application No. 12/950,945, filed on Nov. 19, 2010, now abandoned.
(60) Provisional application No. 61/262,849, filed on Nov. 19, 2009.

(51) Int. Cl. A61M 5/172; A61B 5/145; G06F 19/00; A61M 5/142

(52) U.S. Cl. CPC A61M 5/1723; A61M 5/142; A61B 5/14546; G06F 19/3468; G06F 19/325; A61M 2205/3303; A61M 2205/3553; A61M 2230/201; A61M 2230/205; A61B 2562/0295

(57) ABSTRACT

Medical data provided by a physiological parameter sensor is used for management of the patient's medical condition. Analytes that may be monitored and managed include, but are not limited to, acetyl choline, amylase, bilirubin, cholesterol, chorionic gonadotropin, creatine kinase (e.g., CK-MB), creatine, glucose, glutamine, growth hormones, hormones, ketones, lactate, oxygen, peroxide, prostate-specific antigen, prothrombin, thyroid stimulating hormone, and troponin.

### CROSS-REFERENCE TO RELATED APPLICATIONS

The present application is a divisional of U.S. patent application Ser. No. 12/950,945, filed Nov. 19, 2010, which claims priority to U.S. Provisional Application No. 61/262,849, filed Nov. 19, 2009, both of which are incorporated herein by reference in their entireties for all purposes.

### BACKGROUND

The present invention relates in general to medical data and report generation for medical data and more particularly, to a system and method configured to provide for automated and less costly medical data transfer and to more rapidly and efficiently provide reports from medical data for use by a health care provider during examination of a patient.

Diabetes mellitus, or simply, “diabetes,” is an incurable chronic disease. Type 1 diabetics must manage their diabetes by taking insulin to compensate for the rise in blood glucose that follows food consumption. Type 1 diabetes management works to prevent hyperglycemia, or high blood glucose, while especially averting the consequences of hypoglycemia, or low blood glucose, from over-aggressive or incorrect insulin dosing. Poor diabetes management can manifest in acute symptoms, such as loss of consciousness, or through chronic conditions, including cardiovascular disease, retinopathy, neuropathy, and nephropathy. Effective diabetes management requires effort.

Many different ways exist to assist in monitoring and managing one's glucose levels. Health care maintenance systems based on the use of a hand held device are often used. These devices are configured to record patient data such as blood glucose data. Additionally, it is known that such data can be uploaded to a remote server for storage of large quantities of medical data and later access to it by third parties, such as health care providers (HCP). Examples are Google Health and Microsoft HealthVault™. At the remote server location or elsewhere, blood glucose test results can be matched with quantitative information on medication, meals, or other factors, such as exercise.

Medical sensors can generate large quantities of useful information about a physiological parameter or parameters of a patient. That information, when processed, organized, and reported in particular ways, can be highly beneficial to a health care provider in examining the patient and recommending treatment. The appropriate calculations, organization, and reports of that data can assist in forming rapid, useful, and more accurate evaluations of the information, the patient's history, and the patient's present state and health condition.

For example, analyte monitoring and medication delivery devices are commonly used in the treatment of a patient. One or more samples or analytes from the patient's body tissues is sensed and data is accumulated. A monitor, containing a sensor and a processor, may be used to acquire, accumulate, and process that data. Ultimately a report or reports must be produced from that data for review by the patient and/or his or her health care provider (HCP). In response to the report, one or more medications may be administered to the patient or other course of treatment prescribed. Administration of the medication may be manual by the patient such as self-injection with a syringe, by another person such as a nurse, or by a powered medication administration device, such as an infusion pump, for automatic or continuous delivery. For example, glucose monitors and insulin pumps are commonly used in the treatment and management of type 1 diabetes mellitus.

In the case of diabetes, a blood glucose monitor (BGM) or continuous glucose monitor (CGM) may be used in obtaining data about the glucose level of a patient. Such sensors detect glucose levels through actual analysis of a drop of blood, or through sensing the composition of interstitial tissue. The patient may have a hand held digital device, such as a personal digital assistant (PDA) that is used to receive and store his or her glucose data. This can occur in a number of ways. In the case where the patient draws a drop of blood onto a test strip that is read by a BGM, the data from the BGM may be communicated to the PDA for storage, processing (such as by adding a date and time stamp), and transfer elsewhere. In one case, the BGM is integrated with the PDA (dedicated device). In another case, the glucose data is communicated to the PDA wirelessly or through wired connection. In both cases of the BGM and CGM, various schemes may be used to get measured patient glucose data onto the PDA. The PDA is programmed to process that data and can provide a useful number representation of a glucose level on the screen of the PDA, and can also be instructed to upload the data to a server that may be remote and which may be accessed through the Internet (cloud computing) or by other means. Conveniently, a computerized report can be used to display such measurements and calculations of the measured glucose together and can be used in developing health management recommendations. For example, glucose monitors are programmed to provide recommendations for better blood glucose management in the patient.

For chronic conditions such as type 1 diabetes mellitus, the volume of data that can be created regarding a patient's diabetes-related condition through the use of a glucose monitor operating continuously over a period of time and patient data input regarding meals, exercise, and other activities, tends to be greater than the amount of information that can be readily understood by the patient or a clinician. Data management applications are currently available for processing diabetes data, such as the data just mentioned. Such applications provide reports that include an analysis or multiple analyses of data regarding glucose levels, changes in levels over time, response to insulin delivered, and other information that may be useful to the diabetic patient and his or her heath care provider (HCP). Such analyses often include trends, extrapolations, predictions, alerts, and others.

Hand held devices such as PDAs or dedicated diabetes management devices have limited memories and can only store a certain amount of data before becoming full. If the data is not uploaded or otherwise saved, continued use may cause overwriting of stored data thereby losing some of the medical history of the patient.

Another concern in the collection of glucose data for patients is the expense of saving that data. In the case of uploading it to a remote server, such as one of the commercial server services (Google Health, Microsoft HealthVault™, for example), the data must be transferred by means of some commercially available system. The use of cellular telephone, wireless connection to an Internet Service Provider (ISP), telephone connection, or other services can be relatively costly, especially during the prime usage hours. Additionally, many patients may not be skilled with the use of computers, PDAs, Internet connections, etc. They do not understand the means of connecting to the Internet, uploading data, deleting the uploaded data from the hand held device, and other things. It would provide an advantage to patients if costs for data transfer were lower and if the entire process of data transfer and hand held device were automated.

Despite the importance of effective glucose management, Type 1 diabetics seldom receive direct day-to-day oversight by a physician. Physicians are typically not present at significant metabolic events, blood glucose aberrations, and wide glucose fluctuations. When the patient is present at an office visit and a health care provider can actually observe him or her, such events may not occur. At best, such an office visit provides the health care provider with only a “snapshot” of the patient's diabetes status.

Unfortunately, data management applications and the data generated by analyte monitoring systems are not used by HCPs as widely as desired for a number of reasons. As an example, during a periodic examination of a diabetes patient, it may be of value for the HCP to study the diabetes-related history of the patient since the last visit. In particular, the HCP may desire to see the variations in glucose levels over time, see the relationship of those variations to food intake, exercise, and sleep, see what deliveries of medications were made and their timing, to determine their effects on the patient's glucose levels, and other data. However, providing such data and processed data typically requires a processor, a program to run the processing, a report format, and an output so that the report can be studied by an HCP. This would be the case if the patient were able to accomplish the data download from the monitor, data processing by the program, and printing reports that the patient then presents to the HCP at the time of examination.

In another scenario, the above report generation may also be accomplished by the patient uploading his/her hand held monitor's glucose data to a remote server to which the HCP also has access. The HCP may retrieve the patient's data from the server, process it on a local computer with an application program, and print the results for study by the HCP at the time of the patient's examination. While theoretically this system should be effective, the HCP may not have the necessary time available, nor the assistance to have the report generated by his/her staff. Neither the HCP nor the staff may be sufficiently skilled with computers to obtain the data, process the data, and print reports. Nor may the patient. Persons of ordinary skill are often challenged when connection problems with the Internet or remote servers arise. Thus it would be of value to a patient's diabetes management efforts for the patient's medication history data, as well as helpful reports taken from that data, to be more easily obtainable when needed by a HCP.

Additionally, computers are not typically available in examination rooms during patient visits with a HCP. Also, some HCPs are resistant to learning a number of software applications unique to various medical data device manufacturers that are useful for the manipulation and analysis of the data. Further, some HCPs are unwilling to take the time required to launch a software application and upload data from a medical device (e.g., blood glucose monitor, continuous glucose monitor, insulin pump, and the like) during an office visit. Additionally, different device platforms may require the use of unique cables and connectors, adding clutter and confusion to the medical office environment.

In such cases, an examination must be carried out without the benefit of this accumulated data, and must instead rely on the patient's recollection of the occurrence of events since the last visit, or the patient's notes, in whatever form they may be in.

In one case, a manufacturer has provided a dedicated printer with special upload mechanism. However, this special equipment adds to the clutter of a health care facility, especially in an examination room, and only works with the particular manufacturer's glucose monitor. It is often the case however, that some off-the-shelf computer equipment exists in or near an examination office. Further, an off-the-shelf printer is almost always available, even if a computer is not. It would be valuable to be able to utilize this common equipment in generating medical reports for a patient when needed.

Accordingly, those skilled in the art have recognized a need for a more useful system and method with which a health care provider can obtain patient analyte data and reports for use in a patient examination. A need has also been recognized for minimizing the amount of computer hardware and software that must be obtained, learned, and manipulated to obtain patient health data reports for study by a HCP for an examination. A need has also been recognized for a system and method that allows use of standard printing equipment found in many medical facilities for generating patient data and processed data, and reports for study by a HCP at a patient's examination. And further, a need has been recognized for controlling costs in transferring a patient's medical data to remote servers or elsewhere. The present invention fulfills these needs and others.

### SUMMARY OF THE INVENTION

Briefly and in general terms, the present invention is directed to a data management system comprising a hand held device that stores medical data and transfers that data to a remote server or a health care provider (HCP). In one aspect the stored data in the hand held device is processed into a selected report format and can be forwarded with a selected printer driver for print out at an HCP's office. In another aspect, the report can be processed with the printer driver and the “print” file saved for input to the printer. In yet another aspect, a docking station is used to process the stored data of the hand held device into a selected report format and the docking station is used to select the applicable printer driver or create a print file. In another aspect, the docking station is programmable to automatically transfer the stored data from the docked hand held device to a remote server in batch during a selectable period of time, that period of time selected to result in lower data transfer costs over the communication system selected.

In another aspect, a hand held analyte monitoring device measures or receives blood glucose level data from a blood glucose monitor (BGM) or continuous glucose monitor (CGM). The hand held device includes components and functionality for measuring, storing, and optionally analyzing data relating to one or more measured, targeted or predicted levels of an analyte, such as glucose. The hand held device further includes a data communication interface to facilitate transfer of data or information to another device, such as an intermediate portable data communication, a printer, computer, or the like, as well as printer drivers and other computer-readable instructions for transferring and/or printing data. The hand held device also includes a timing program for automatic, semi-automatic or user-initiated data transfer to a docking station, printer or remote server.

The docking station includes components and functionality for the transfer and analysis of data to and from the hand held device as well as at least one data communication interface for communication with the hand held device. Multiple communication interfaces for the transfer of data from the hand held device to a computer, remote server, printer or the like are provided. In another aspect, the docking station is configured as a charging device that provides a charge to a power source, such as a rechargeable battery in the hand held device. In other aspects, the docking station may include one or more printer drivers, printer management programs, or combinations thereof such that data may be sent directly to a printer to provide for convenient data review, for example to assist users and HCPs during office visits. In yet a further aspect, the docking station is provided with a timing program for automatic, semi-automatic or user-initiated data transfer to a docking station, printer, or remote server.

As utilized in the invention, the docking station provides an interface for data and information transfer from the hand held device to a remote server for manipulation by a user or HCP or to a printer for printing data collected by the hand held device. In further aspects, the docking station includes a variety of features to enhance its capabilities, such as audio speakers, and a display to provide the user with various information.

The other aspects in accordance with the invention, there is provided a method for establishing a connection between a hand held data management device and a docking station, wherein the docking station is configured for interlocking with the hand held management device. The method includes transferring data associated with the hand held device to the docking station. A connection is established between the docking station and a remote server and the data associated with the hand held device is automatically uploaded to the remote server by the docking station. The method may further include executing computer-readable instructions stored on the docking station for driving a printer to accurately print a report selected from a plurality of selectable reports on the docking station. Further, the method may be performed automatically upon mounting the hand held data management device with the docking station. For example, data collected and stored on the hand held device may be automatically uploaded to a remote server upon connecting the hand held device with the docking station.

In yet another aspect in accordance with a method, an automatic data upload of stored data in the hand held data management device is performed on a timed basis, where a timing program runs on the hand held device or the docking station. In response to a start command being activated in the timing program, the current time would be detected and compared to a predetermined upload time. If the same, a request for data would be sent to the hand held data management device by a wireless or wired connection, or through a USB, Firewire, or other data transfer port. On receipt of data from the hand held data management device, an Internet connection is established between the docking station and a remote server according to predetermined access instructions in a communications program resident on the cradle and the data uploaded to the server.

In a more detailed aspect, both the timing program and communications program are resident on the hand held data management device, with the docking station serving only as a communications conduit for establishing a data connection to the remote server.

In a further more detailed aspect, the docking station is also provided with a program of computer-readable instructions for determining which data have been previously uploaded when a request for a further upload to a remote server is made, so that only new data are requested from the hand held data management device and transmitted to the remote server. This can be accomplished by storing information concerning the last data upload in memory on the docking station or the hand held device, or by querying the remote server for the last data record sent. If the last data record information is stored on the hand held device, it will respond to a data upload inquiry from the docking station only by transmitting data not previously provided. Memory storage on the docking station can also be provided for tracking previous alarms, error and status messages, and for providing an audible or visible signal for new alarms or if an error in transmission or receipt of data occurs.

In yet a further more detailed aspect in accordance with the invention, the hand held data management device is provided with functionality sufficient to allow printing of data directly from the hand held device, or from a data storage device coupled to the hand held device. In another aspect such data storage device is removable and portable, such as a flash memory card or a flash drive. In the former case, the hand held data management device is configured to allow transmission of data to a printer and the printing thereof using a common printer driver and management program, such as those native to standard operating systems for computers, such as Microsoft Windows®.

Various features and advantages of the invention will become more apparent by the following detailed description of several embodiments thereof with reference to the attached drawings, of which:

### BRIEF DESCRIPTION OF THE DRAWINGS

FIG. 1 is a block diagram of a system for monitoring and reporting a medical condition having a hand held device used for managing that medical condition and in which is stored patient medical data, the hand held device being shown with a wireless connection to a remote server, the remote server also being accessible to the patient's health care provider, the office of whom includes a computer and a printer for providing reports to the HCP derived from the patient's medical data during an examination of the patient;

FIG. 2 is a block diagram of a system similar to FIG. 1 but in this case showing the wireless interaction of the patient's hand held device with the HCP's computer and/or the printer to cause either to generate the medical condition reports for review by the HCP during or before the patient's examination;

FIG. 3 is also a block diagram of a system similar to FIG. 1 but in this case, the HCP's office includes a docking station (sometimes referred to as a cradle) for receiving the hand held device of the patient, downloading patient medical data from that docked device, formatting it into a selected report, selecting a printer driver, and communicating the processed medical data report to either the HCP's computer or the printer, or both;

FIG. 4 is another block diagram of a system similar to FIG. 1 but in this case, the patient's hand held device is configured to accept and use a portable removable memory device, such as a SanDisk™ flash memory card, on which the hand held creates a report usable by either the HCP's computer or the HCP's printer, or both, the figure showing the memory card being removed from the hand held device at the HCP's office and being inserted into the computer or the printer for reading and for printing the desired report of the patient's medical data for use by the HCP in the patient's examination;

FIG. 5 is a block/flow diagram of one embodiment of a patient's hand held device in which multiple application programs exist, one of which is for preparing a medical data report, the hand held interface permitting a printer selection to be made, a report selection to be made, in which case the processor then accesses the stored medical data, formats it in accordance with the selected report, accesses the correct printer driver, configures the data accordingly, and communicates that report to the communication unit of the hand held device for printing by an HCP printer;

FIG. 6 is a block diagram of components of a system in accordance with aspects of the invention including a patient device, a docking station, a printer, memory card, and others, with a host server;

FIG. 7 shows a docking station or “cradle” in block diagram form with various top level functions indicated within the box indicating the docking station;

FIG. 8 is a flow diagram illustrating a method of data analysis and transfer from a monitoring device to a cradle and/or a remote server, according to an embodiment; and

FIG. 9 is a flow diagram illustrating a method of data transfer to a printer for preparing a medical data report, showing the direct transfer of data to a computer, to a removable memory device, or directly to a printer, according to certain embodiments.

### DETAILED DESCRIPTION OF THE PREFERRED EMBODIMENTS

Reference will now be made in more detail to the drawings, wherein like reference numerals refer to like elements throughout. Well-known functions or constructions will not be described in detail so as to avoid obscuring the description with unnecessary detail. It should be noted that in the drawings, the dimensions of the features are not intended to be to true scale and may be exaggerated for the sake of allowing a clearer understanding.

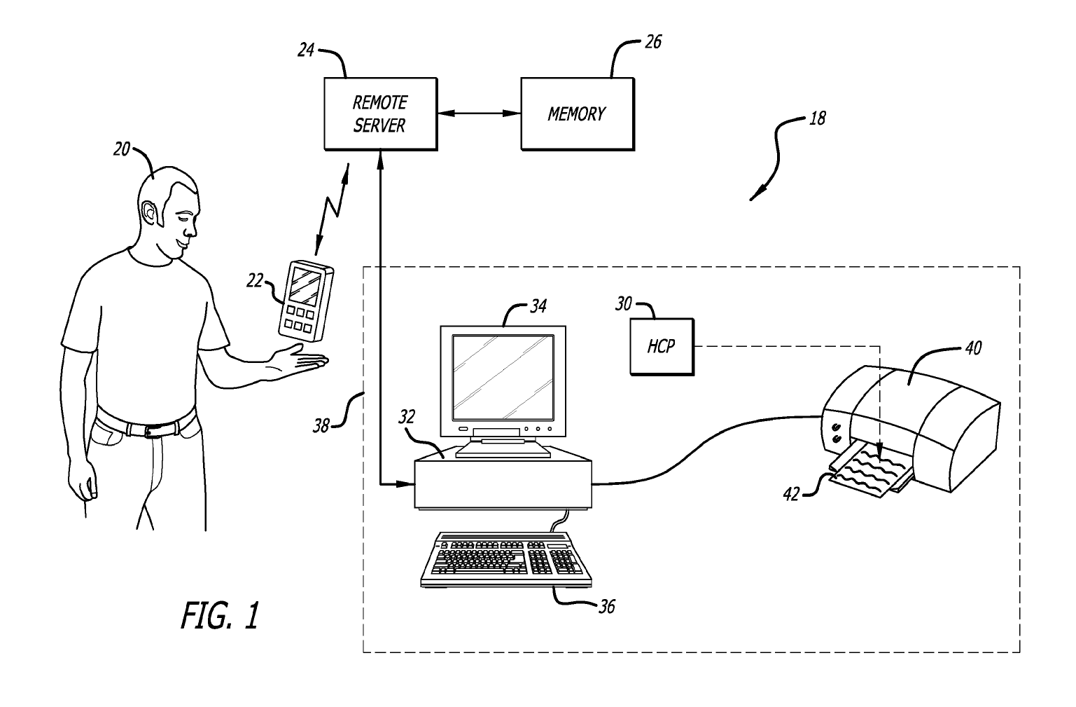

Turning now to FIG. 1, an overall block diagram is presented of a data management system 18 in which the medical data of a patient 20 is uploaded from a hand held device 22 to a remote server 24 having a memory 26 for storage of large amounts of patient medical data. In this embodiment, the hand held device and the server are communicating with each other via wireless link 28 directly to the server; however, this is for ease of illustration only. It is likely that other data receivers/transmitters would intervene. Additionally a wired connection along the route to and from the server may exist.

In some cases, such connections between the hand held device 22 and the remote server 24 are used to provide for the rapid and real-time upload of patient medical data. Such systems using real time communications may incur relatively higher communication costs if they communicate during peak usage hours. This is also discussed elsewhere herein. Rates for data transfer are typically much higher at the peak volume data transfer times of the day, such as during business hours, than they are during the middle of the night when many people are sleeping. Such rates ($/minute) may be lowered somewhat depending on the terms of a usage contract. As is provided in an embodiment below, communication at low usage times is employed to reduce costs.

Continuing with FIG. 1, the remote server 24 may also be accessed on the patient's behalf by a health care provider (HCP) 30. For example, the HCP 30 may connect to the remote server 24 with a local personal computer 32 (shown with a display 34 and keyboard 36), or other similarly functioning computing device. The local personal computer 32 includes a memory, processor, and an application program (not specifically shown) that enables the HCP to identify the patient and the desired data to the remote server. The remote server 24 in turn is able to locate the requested data in the memory 26, retrieve it, and download it to the HCP computer. The remote server may or may not offer the ability to run programs on it to create reports from the patient's stored data and download such reports to the HCP personal computer. If this is not available on the remote server, the HCP's personal computer may have a report generating program that can store the patient's data, process it, and create the necessary printed reports from it, all at the HCP's office 38. Located at the HCP's office 38 is an off-the-shelf (OTS) printer 40, on which the desired report 42 can be printed for review 43 by the HCP during examination of the patient.

An “off-the-shelf” (OTS) printer is one that is commercially and widely available to the general public and for which the “printer driver” can be readily obtained.

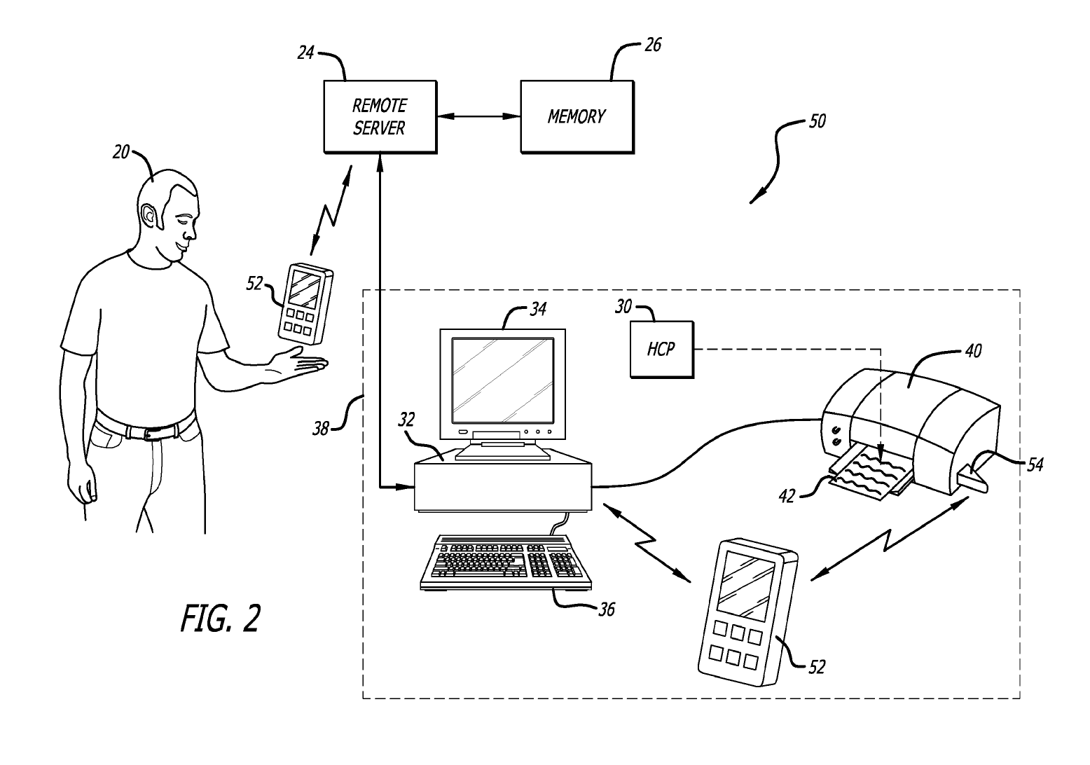

In FIG. 2, a different medical data management system 50 for the transfer of medical data is provided. The patient 20 has a hand held device 52 that stores patient-specific medical data. The present system 50 is usable for diabetes management systems and the hand held device 52 receives and stores glucose data and other diabetes-related data about the patient. The hand held device 52 may include a strip reader to analyze a drop of blood for glucose content. The strip reader may be integral with the hand held device or may be connected with it. The additional data may include, but is not limited to, insulin delivery times and amounts, exercise times, meal times, and carbohydrate content. As in FIG. 1, there is a HCP 30, a HCP's office with computer equipment, and a remote server 24 and memory 26.

In this case, the hand held device 52 has an integral wireless communication system and an embedded program that enables the processor of the hand held device to prepare medical data reports 42 for use by the HCP 30. In particular, the hand held device would receive a report selection and a printer driver selection, although a default printer driver may exist. The processor would then retrieve the relevant medical data from the memory of the hand held device, process it to prepare the report, and then wirelessly communicate the report as needed. Referring again to FIG. 2, the report printing could be done at the HCP's office 38. In one arrangement, if the HCP computer 32 or LAN network is wireless configured and capable, it may receive the report wirelessly from the hand held device, process it, and cause the printer 40 to print the report 42. The HCP 30 would then be able to review the report as is shown by the dashed line in FIG. 2. In another embodiment, the printer may include a wireless adapter 54 and be able to receive the report from the hand held 52 for printing 42. The hand held device report printing program would wirelessly contact the printer, identify the printer type, select the correct printer driver from a data base of stored printer drivers in the hand held, perform the necessary negotiation with the printer, and have the report printed.

Although a wireless connection is shown with the computer 32 and/or the printer 40 in FIG. 2, a wired, infrared, or other connection may be usable, depending on the hardware and software available at the HCP's office 38. It is important to note that in this case, the HCP's computer 32 is not needed if the printer includes a wireless adapter 54. This feature allows for use of the most current patient medical data, and the rapid and more convenient generation of the report 42 without the need for the HCP 30 to connect with the remote server 24, run an application program on the HCP's computer 32 and print a report. The present system 50 permits the preparation and printing of the report much more easily and conveniently.

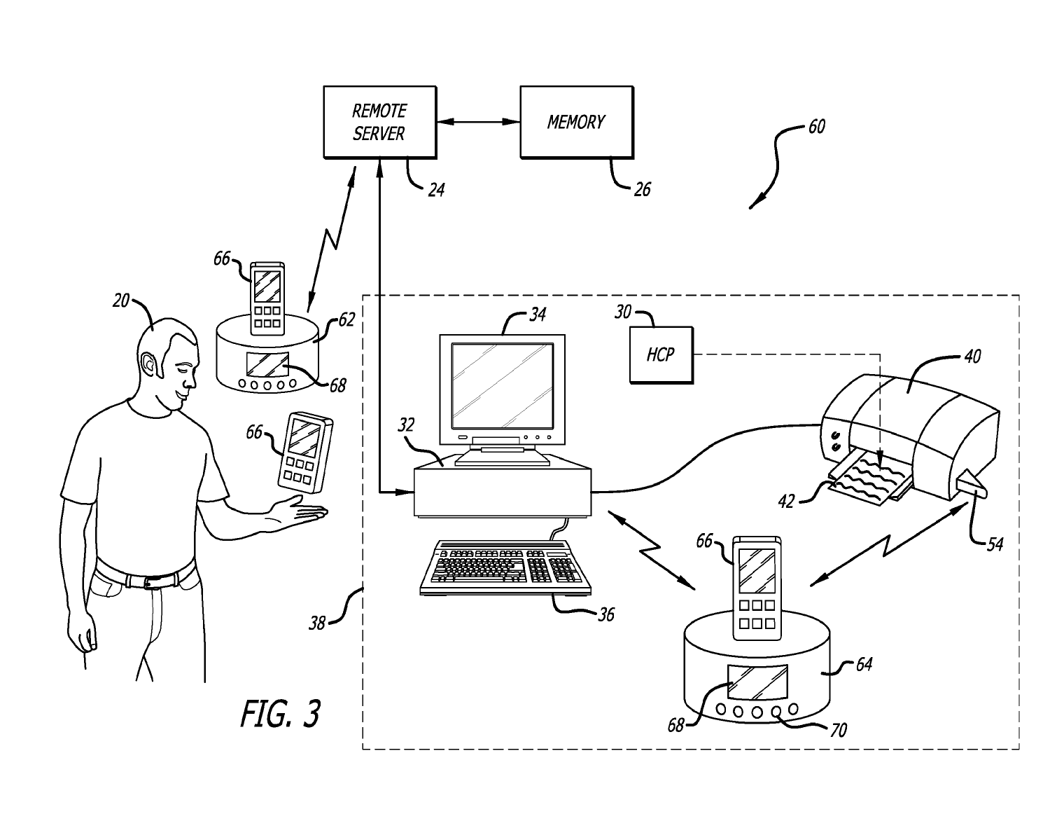

Referring now to FIG. 3, a different data management system 60 is shown in which docking stations 62 and 64 are used. As shown at the left side of the figure, the patient 20 has a handheld device 66 that in this case is used in a docking station 62 to communicate with the remote server 24 and remote memory 26. The docking station 62 can be programmed to automatically contact the remote server 24 at the hours of the day when data transfer is least expensive. Alternatively, if a contract has been entered into with an ISP or other data communication company, and that contract provides for the lowest rates when data is communicated over its data communication equipment at a particular time period, the docking station may be programmed to do so during that time period.

The docking station 62 in this embodiment is programmed to retrieve the medical data from the patient hand held device 66 that has been mounted into the docking station 62, and at some later time, or at the time of download from the hand held device, automatically forward that data to the remote server 24, as discussed above. At the same time, the docking station 62 may recharge the battery in the hand held device 66 as well as erase the data from the hand held that has been successfully transferred to the docking station and/or the remote server. The docking station may also be programmed to present medical data on a display 68 or to prepare reports for printing, as is discussed below. The docking station can also present indicators to the patient of when its activities are complete, or when an error exists. For example, the docking station may indicate by a green light that the battery recharging of the hand held device is complete. It may also indicate by red light that the charging is not complete but is ongoing. It may indicate by a “fuel gauge” on the display 68 the progress of the data upload to the remote server, for example.

The docking station programming of the automatic communication routine for the upload of all data in the docking station to the remote server may occur through various data transfer systems. For example, the patient may base his or her decision on which way to communicate with the remote server based solely on cost, since connection and upload are automatic. For example, the patient may use a wired or wireless router with an Internet Service Provider to transfer the data, or a cellular telephone connection if the hand held takes the form of a “smart” phone, or a wired telephone connection, or other. This feature enables a patient to control his or her costs yet still get the important medical data to the remote server.

Another advantage of this docking station 62 is that no data bases of printer drivers or report configurations are needed within the hand held device 66. All of these data may be located within the docking station, which may be used for the existing hand held device and replacement hand held devices. This feature will result in a lower cost hand held device.

In the embodiment of FIG. 3, once the hand held device 66 is placed in the docking station 64, the docking station then takes control over the hand held device. Data is transferred, the battery is recharged, and the hand held's memory is erased.

The arrangement of FIG. 3 presents even further advantages at the HCP office 38. As was discussed in the background section, a convenient and easy way to obtain medical data reports about a patient at the time of examination is needed. The docking station 64 at the HCP office 38 fulfills this need. The patient need only bring his or her hand held medical data management device 66 to the HCP's office 38, mount it into the docking station 64 at that location, and the HCP staff, the HCP 30, or the patient can proceed to select the report desired by the HCP, select the correct printer driver for the HCP's printer 40, press “PRINT” and the docking station will organize the report for the medical data in the hand held device 66, format it, and apply the printer driver to it. The docking station 64 will already be set up for wired or wireless communication with the HCP's computer 32 or directly with the HCP's printer 40 and the report 42 will be printed. With this embodiment, there is no need for the HCP's staff to attempt to connect to the remote server 24, find and extract necessary data, and run the report themselves.

The programming of the docking station 64 in this embodiment causes it to communicate the patient medical data in the hand held device 66 to the HCP computer 32 or printer 40. In one embodiment, the HCP computer is programmed to process the patient data and generate one or more reports 42 for review by the HCP during an examination of the patient. However, if the HCP does not have a suitably programmed computer that will process the patient medical data, or if the program on the computer 32 is corrupted, or the computer is otherwise engaged, the docking station 64 in accordance with aspects of the invention is programmed to process the patient data into the reports desired, and communicate directly with the printer to cause the printer to print those reports 42. In such case, the docking station includes the appropriate printer driver or has a list of them from which the HCP may select for his or her printer 40 by reviewing the display 68 and manipulating the control buttons or keys 70, as needed.

Another aspect in accordance with the invention is a program located either in the hand held device 66, in the docking station 62 or 64, in the remote server 24, or in a computer 32, which automatically initiates upload of the data from the hand held device to be analyzed. The timing of this initiation could be real-time, that is at the time that the hand held device acquires a data point (for instance, a glucose reading), or the timing could be periodic such as every day at 2 am or once per week on Sunday at 3 am, as examples only. That is, the upload could automatically occur periodically, though not necessarily real-time, at times when it communications may be inexpensive. This mechanism would be less costly and convenient to the patients.

The upload initiation is performed by simple code that compares the device time with a preset time. When the device time matches this preset time, upload is initiated. The preset time may be adjusted manually via a user interface or preset in the program code.

If the upload initiation program is located in the hand held device, then the program would simply attempt to establish and perform communication for which it has be configured. For instance, if it was configured for wireless 3G or pager communication with a phone network to a server, this is how the upload would occur. If it was configured for wireless communication via a standard wireless router to an internet IP address, then this is how the upload would occur. If the upload failed for any reason, the program would reschedule retry attempts within an appropriate window of time, say when communication rates are still cheap, and/or would just retry at the next preset scheduled time.

In an alternative embodiment, the upload initiation program is located in the docking station, or a similar device that may communicate with the hand held wirelessly such as a smart router. The program may have two levels of timing; one where it queries the hand held device for data at a preset time or periodicity, and another where it sends the data to a remote server at another preset time. These times may be determined to support convenience, power conservation (for instance, in the hand held) or cost. For instance, the program could be set to query the hand held device for any new data every 4 hours, and buffer the data. Then the program may send the accumulated data at a different preset time, say once per week on Sunday at 3 am. In another aspect of this invention, the hand held and the docking station may synchronize their communication times, for instance, so that the hand held would save power by only transitioning to a higher power mode when communication is planned.

In another embodiment, the initiation program is located in the remote server that is the destination for the data. The program could query the hand held directly for the data upload, which may only be practical when the hand held is in constant communication such as a 3G enabled device. Alternatively, the program could query a docking station to upload the data, for instance at a preset time. This embodiment assumes that the docking station has buffered uploaded data, where the hand held data upload was initiated by a program located either in the hand held or the docking station as discussed above.

As used herein, “batch processing” refers to a mode of data processing in which data is gathered over a period of time and aggregated for subsequent processing. As used herein, “flash memory” is a non-volatile computer storage chip that can be electrically erased and reprogrammed. Flash drives and pen drives are USB storage devices based on flash memory. Flash memory is primarily used in memory cards, USB flash drives, MP3 players, and solid-state drives for general storage and transfer of data between computers and other digital products. It is erased and programmed in large blocks. Flash memory provides non-volatile, solid-state data storage. Example applications include PDAs (personal digital assistants), laptop computers, digital audio players, digital cameras, and mobile phones. Since flash memory is non-volatile, no power is needed to maintain the information stored in the chip and it is portable, by not needing a power source (portable by itself). In addition, flash memory offers fast read access times. Other types of memory that exist now or that are created in the future may also be used.

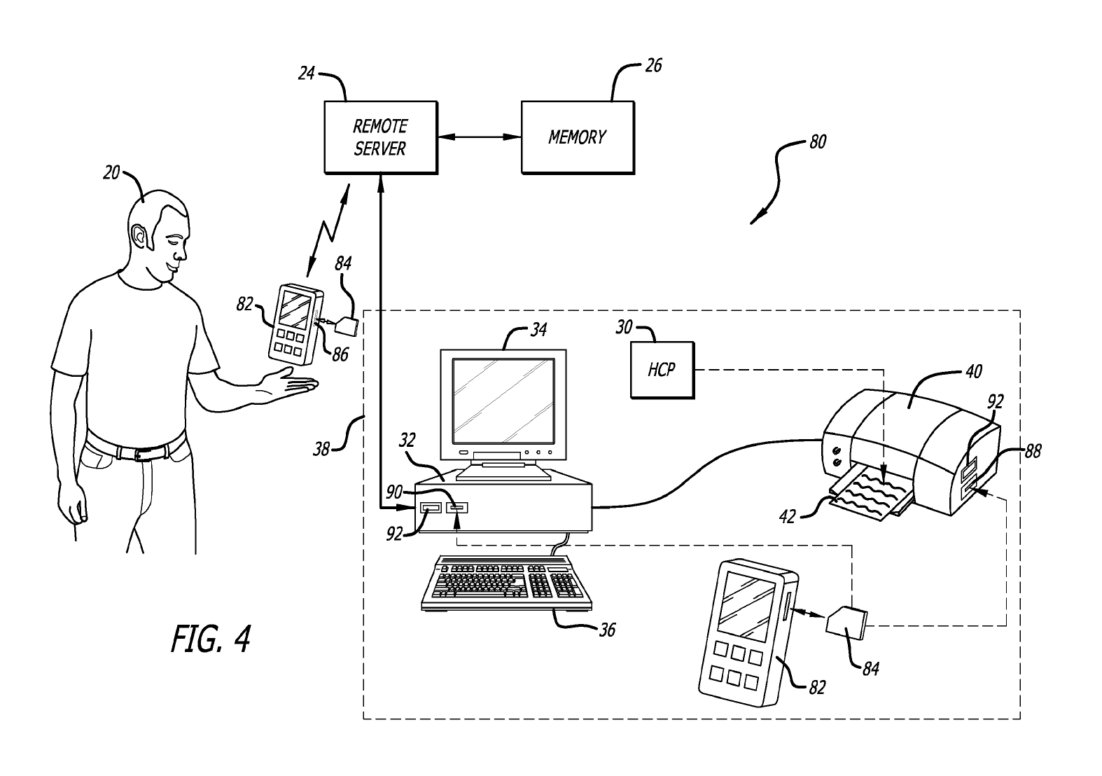

Turning now to FIG. 4, another data management system 80 is shown comprising a data management hand held device 82 with a removable memory device 84. In this system, the hand held device includes the programming and data bases wherein the user may select a report form and the internal processor will retrieve, process, and organize the stored data into the format of the selected form. The processor will also accept the user's input as to which printer driver to apply and will prepare a printable report on the removable memory device 84. Then, when in the HCP's office, the patient or HCP or staff can remove the memory device from the hand held device, and read it by mounting the removable memory device 84 either in the computer 32 or the printer 40. In this case, the hand held device 82 has a removable memory mounting slot 86 into which the removable memory device is pushed so that operation of it is possible. When the memory device is to be used, it is either ejected from the slot 86 or is manually pulled out and mounted into a similar slot on another compatible device, such as the memory card slot 88 on the HCP's printer 40. The computer 32 also has a memory card slot 90 in this embodiment. Also in this embodiment, the computer and printer also have USB connectors for receiving a flash memory device by USB. The hand held device may also be configured to use USB flash, in another embodiment.

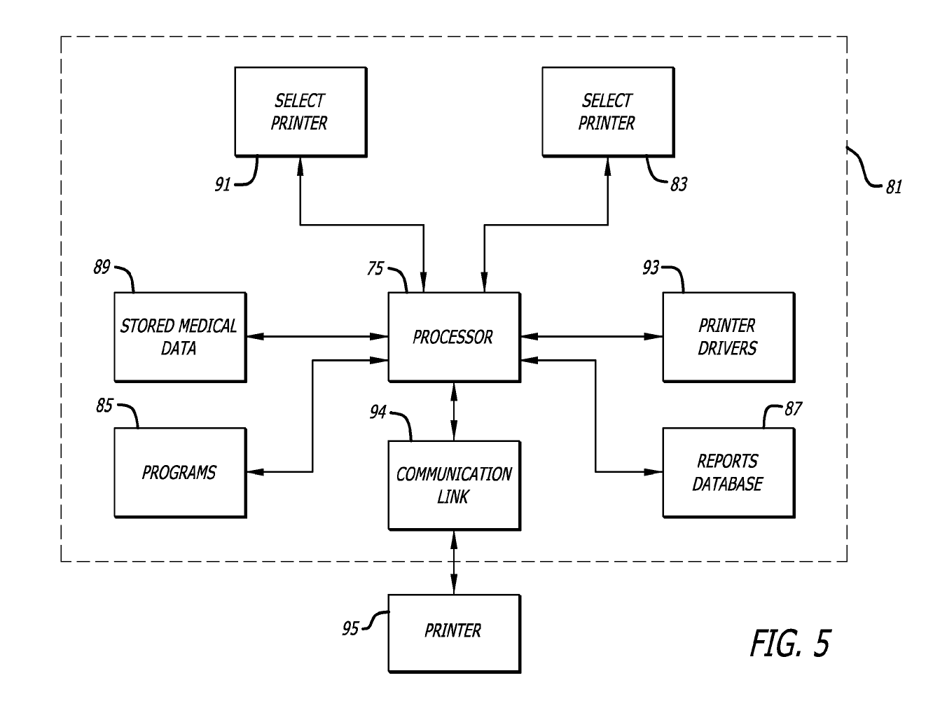

FIG. 5 presents a block diagram/flow chart of the process of preparing a report, as discussed above. The hand held device is indicated in dashed lines 81. The user selects a particular report 83, in response to which the processor 75, through the use of the appropriate program 85, retrieves the selected report format from the reports data base 87. The processor then retrieves the necessary medical data 89 to complete the selected report. The user also selects a printer driver 91 for the printer on which the report is to be printed. The processor retrieves the selected printer driver 93 from a memory and combines the report with the printer driver information. The processor then communicates 94 the complete report with printer driver information to a device, which in this case is a printer 95. However, that device could also be a wireless adapter, a flash memory device, a computer connection, or other.

Some embodiments of the invention provide the user a single portable or hand held electronic device with analyte detection, data storage, and data transfer to other data storage or processing (versus having multiple stand-alone devices performing these functions). Some embodiments of the invention also allow the user to have a single portable electronic device which is not restricted to analyte detection, but can be used with other functionality, such as cellular phone or personal digital assistant functions. The use of portable electronic devices continues to increase and more and more features are embedded into the portable electronic devices. Some embodiments of the invention add a new dimension or feature to the portable electronic device for use in personal health and disease management. The glucose measurement system can improve the data collection process by allowing a single portable electronic device to transmit or receive data to and from the physician and the individual via wireless or wired connections such as Bluetooth, IR, USB, and others. The glucose sensing system can also improve the time to administer medical therapies, assess compliance and provide data for insurance providers, individuals, and physicians.

In further detail, in the case where the portable electronic device includes an embedded glucose monitor having a port for accepting and reading blood glucose strips, the strips are inserted into the reader of the hand held device, are analyzed by the reader, and the data representative of the glucose data from that reading are stored in the memory of the hand held device. In most cases, a time and date stamp is attached to that data. The integration of the glucose test strip reader into the hand held device provides the individual patient with dual functionality in a single portable electronic device (versus multiple devices). In disease management, the majority of the type 1 diabetics are asked to monitor diet and fitness, and in some cases engage in a weight loss regimen combined with the common need for diabetics to test their blood sugar (blood glucose measurement). The hand held device in accordance with aspects of the invention provide the patient or user with a single, well-rounded tool to control his or her health. As shown above, the medical data stored can be sent to the individual's health care provider for diagnostic, feedback and treatment, and medical therapies.

Some embodiments of the invention can be used by cellular phone manufacturers, individuals who do not want to carry multiple portable electronic devices with them (this allows for a multifunctional single device), physicians whose patients are diabetic (specifically type 2 diabetes to monitor diet compliance), and physicians who perform weight loss surgeries to help monitor dietary compliance.

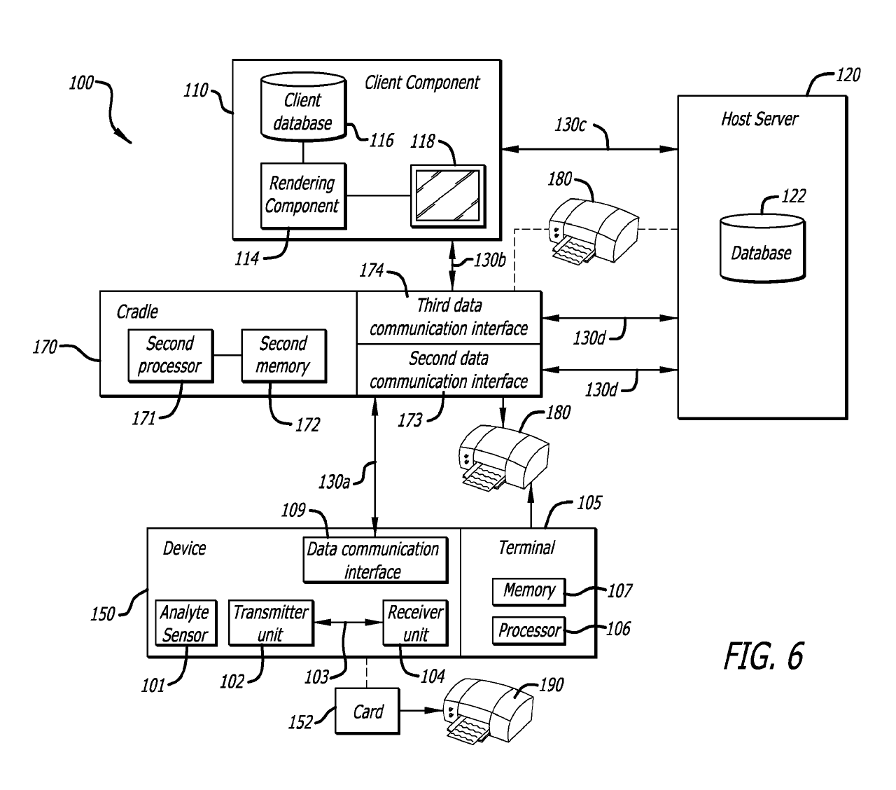

With reference now to FIG. 6, a biological monitoring system 100 is illustrated in connection with client and remote servers. The system 100 may be, according to an embodiment, an analyte monitoring system, such as a glucose monitoring system. However, the system 100 is not limited to such an embodiment. For example, the analyte monitoring device 150 of system 100 shown in FIG. 6 may instead be or include a different medical device, such as a drug delivery device that stores or otherwise acts upon medically relevant data, such as an insulin infusion pump that stores or otherwise acts upon data relating to blood or interstitial glucose measurements, carbohydrate intake values and other data of interest in diabetes management. However, for ease of reference, the device 150 shall be referred to herein as an analyte monitoring device (but the term shall be generally understood to extend to other kinds of devices, such as drug delivery devices).

Analytes that may be monitored and managed by the system 100 include, but are not limited to, acetyl choline, amylase, bilirubin, cholesterol, chorionic gonadotropin, creatine kinase (e.g., CK-MB), creatine, glucose, glutamine, growth hormones, hormones, ketones, lactate, oxygen, peroxide, prostate-specific antigen, prothrombin, thyroid stimulating hormone, and troponin. The concentration of drugs, such as, for example, antibiotics (e.g., gentamicin, vancomycin, and the like), digitoxin, digoxin, drugs of abuse, theophylline, and warfarin, may also be monitored. The invention is particularly well-suited to use with devices for storing, using or transmitting data relating to automated, continuous or otherwise regular measurement of a medically relevant analyte, such as blood or interstitial glucose.

Thus, as shown in FIG. 6, the system 100 may include an analyte monitoring device 150, which comprises an analyte sensor 101, a transmitter unit 102 coupled to the sensor 101, and a primary receiver unit 104 which is configured to communicate with the transmitter unit 102 via a communication link 103.

The analyte monitoring system optionally includes a further, separate data processing terminal 105, which may include at least one processor 106 and at least one memory 107 for storage of data. Data processing terminal 105 may include an infusion device such as an insulin infusion pump or the like, which may be configured to administer insulin to patients, and which may be configured to communicate with the receiver unit 104 for receiving, among others, the measured analyte level. Alternatively, the receiver unit 104 may be configured to integrate an infusion device therein so that the receiver unit 104 is configured to administer insulin therapy to patients, for example, for administering and modifying basal profiles, as well as for determining appropriate boluses for administration based on, among others, the detected analyte levels received from the transmitter unit 102. The data processing terminal 105 may include a memory 107 or be in connection with a data network or a database (not shown) for storing, retrieving, and updating data corresponding to the detected analyte level of the user.

Accordingly, the analyte monitoring device 150 includes an analyte sensor 101, a processor 106, a memory 107, and a data communication interface 109 operatively coupled to the transmitter unit 102 or the data processing terminal 105, including the processor 106 and memory 107. Generally, memory 107 provides for storage of data relating to one or more measured, targeted, or predicted levels of the analyte, and the data is typically contained in a report format. The data communication interface 109 facilitates transfer of data or information to another device, such as the cradle 170, the printer 180, the client component 110, the remote server 120 or others.

Additional detailed description of a continuous analyte monitoring system and its various components including the functional descriptions of the transmitter are provided in U.S. Pat. No. 6,175,752 issued Jan. 16, 2001, entitled “Analyte Monitoring Device and Methods of Use,” in U.S. application Ser. No. 10/745,878 filed Dec. 26, 2003, entitled “Continuous Glucose Monitoring System and Methods of Use,” U.S. application Ser. No. 12/024,101 filed Jan. 31, 2008, entitled “Method and System for Determining Analyte Levels,” and in U.S. Provisional Application No. 61/184,234 filed Jun. 4, 2009, entitled “Failure Recovery Methods of Corrupted Device or During Software Downloads and Preservation of User and Manufacturing Data,” each assigned to the Assignee of the present application and incorporated herein by reference in their entireties.

In an exemplary aspect, system 100 further includes a cradle 170 for interlocking with the analyte monitoring device 150. Either or both of the cradle 170 and the analyte monitoring device 150 may be configured with functionality to store, manipulate, analyze and transfer data, with device 150 including further functionality for measuring data relating to one or more measured, targeted or predicted levels of an analyte.

As shown in FIG. 6, the analyte monitoring device 150 is in operable communication with the cradle 170 via A data communication interface 130a. The cradle 170 includes a second processor 171, a second memory 172, and a second data communication interface 173, where the second data communication interface 173 is operatively coupled to the first data communication interface 109 for transmission of data from the analyte monitoring device 150 to the cradle 170 via the operative connection 130a.

The cradle 170 may further include additional data communication interfaces to facilitate simultaneous connectivity with additional components. For example, the cradle 170 may include a third data communication interface 174 for simultaneous operative coupling with a client component 110 (e.g., a computer or personal digital assistant (PDA)), a printer 180 or a remote server 120. One of skill in the art will understand that the cradle 170 may be configured with as many additional data communication interfaces as necessary to facilitate simultaneous operative coupling with one or more of the client component 110, the printer 180, the remote server 120, or any additional devices.

Where the cradle 170 is present, the first memory 107 and/or the second memory 172 thereof further includes stored instructions. When executed by the first processor 106 or the second processor 171 (automatically, according to a timing program, or on receipt of a user entered command), the instructions cause first processor 106 or second processor 171 to detect a connection between the analyte monitoring device 150 and the cradle 170, identify the connected analyte monitoring device 150, and transfer data associated with the analyte monitoring device 150 to the cradle 170.

Further, the second memory 172 of the cradle 170 further includes stored computer-readable instructions which, when executed, causes the cradle 170 to connect with a remote server 120, directly via connection 130d or indirectly through a client component 110 via connections 130b and 130c, through second data communication interface 174, and upload the data to the server. Additionally, in an exemplary aspect, the second memory 172, further includes a printer driver and computer-readable instructions for printer management stored therein such that a report may be printed on any printer without the need for any additional devices or software.

Only one sensor 101, transmitter unit 102, communication link 103, and data processing terminal 105 are shown in the system 100 illustrated in FIG. 6. However, the system 100 may extend to a multi-component and/or multi-device environment, each component and device is configured to be uniquely identified by each of the other components and devices in the system so that communication conflict is readily resolved between the various components and devices within system 100.

Communication links between components of the system 100 may be any suitable communication protocol for transferring data, including one or more of an Ethernet connection, RF communication protocol, an infrared communication protocol, a Bluetooth enabled communication protocol, an 802.11x wireless communication protocol, an equivalent wireless communication protocol, a serial or USB connection, or other.

Operation of the invention will be described with respect to a single analyte monitoring device 150 linked to the client component 110 or printer 180, optionally via the cradle 170, but the invention will be understood not to be limited to such devices and links. In one embodiment, the analyte monitoring device 150 is connected to the client component 110 via the communication links 130a-130b via the cradle 170, whereupon the medical data generated by the monitor 150 is uploaded to and stored in the client database 118 or transferred directly to the remote server 120 via the connection 130a. In an alternative embodiment, the analyte monitoring device 150 is connected to the remote server 120 via the communication link 130a via the cradle 170, whereupon the medical data are uploaded to and stored in a database 122 or are otherwise manipulated by the server 120.

Conveniently, medical data generated by monitoring device 150 may be uploaded to the cradle 170 for transmission to the printer 180 or, where the analyte monitoring device 150 is provided with printer drivers, such data may be transmitted directly to the printer 180. Alternatively, the analyte monitoring device 150 may optionally be provided with a removable storage device 152, such as a memory card or a USB drive, for insertion into a corresponding slot in the printer 190 for direct printing of stored data therefrom. The transmission of medical data may be continuous, automatic, at predetermined time intervals, at predetermined times, or upon command by the patient or an external user.

Preferably, the transmission of data occurs at predetermined time intervals according to a timing program resident on the analyte monitoring device 150 and/or the cradle 170. The computer-readable instructions comprising the timing program may provide for a variety of different settings, including automatic data transfer upon establishment of a connection between the monitoring device 150 and the cradle 170, transfer at a predetermined time of day or date, or transfer upon entry of a user request for upload, following a connection being established between the analyte monitoring device 150 and the cradle 170. For display of upload status information and entry of user commands, the analyte monitoring device 150, the cradle 170, or both may include a user interface; e.g., a LED display and keypad.

Within the cradle 170 is also provided a memory 172 for storage of communication protocols for uploading data to a remote server which may include, without limitation. Internet access instructions for the system, passwords, and the like.

Users of the client component 110 or the remote server 120 may access the medical data for processing by the report software application 112 or a similar application of the remote server 120 to obtain and display, for example, different calculations and/or representations related to the medical data. Similarly, the user of cradle 170 may access medical data, and use report software application 112 or similar application of remote server 120 to process the medical data, or use a software application stored on the cradle 170 or analyte monitoring device. The processing of the medical data may include various operations, such as, but not limited to, determining medicinal dosage, calculating various chemical and/or biological attributes related to the patient, such as glucose or blood-sugar levels, and preparing graphical or other representations of the medical data. Processed data may be stored on the client component 110 in the database 118, or on the remote server 120 in the database 122.

Again referring to FIG. 6, the client component 110, when present, maybe embodied in a computing device, such as a user's personal computer, laptop, and/or handheld device such as a PDA or smart phone. The client component 110 typically includes a graphical user interface (GUI) rendering component 114 for providing an interface, such as a graphical user interface (GUI) 116, that allows for user-interaction related to components displayed on the GUI 116 and for populating the GUI 116 based upon information received from the host server component 120 or the monitoring device 150 and/or the cradle 170. The GUI rendering component 114 may provide GUI 116 with user-controllable features to allow the user to view, enter, upload, download, or otherwise manipulate and access data and information. Web-based application software and other client software may be stored in memory and may be executed by one or more processors of the client component 110.

The GUI rendering component 114 receives the medical data and the processed information and populates the GUI 116 with the either or both sets of information (all “medical information”). The user is able to view the medical information through user-interaction on the GUI 116. For example, multiple windows, boxes, icons, or other GUI components may be available for the user to formulate a desired request or obtain desired medical information. The user of the client component 110 is able to save accessed medical information on the client database 118 for later access thereto. Alternatively, as discussed elsewhere herein, the cradle 170 may include similar functionality to store and display medical data.

According to an embodiment, the analyte monitoring system as well as other related components, such as the client component 110 and the remote server 120 may be used to implement a computer-based data management system known as the CoPilot™ Health Management System (CoPilot). The CoPilot system is a personal computer (PC or portable or handheld appliance)—based software application that permits people with diabetes, their healthcare team, and caregivers to upload data from FreeStyle™ and Precision Xtra™ blood glucose monitoring systems, as well as Navigator™ CGM (and generally from several other commercially available blood glucose meters and insulin pumps) into the CoPilot application.

The CoPilot system provides graphs and other software tools for people with diabetes and their healthcare professionals (HCPs) to help evaluate and analyze medical information such as glucose readings, carbohydrate intake, insulin dosage, exercise and other diabetes-related factors uploaded from devices or manually entered into the system. The system can help identify trends that can be used to educate persons with diabetes to improve their glucose control, for example.

Additional detailed description of the above-described PC-based software application for healthcare management and its various features and functionality are provided in U.S. patent application Ser. No. 11/146,897 (Publication No. 2006/0010098) filed Jun. 6, 2005, entitled “Diabetes Care Report generation Architecture and Data Management System,” and U.S. Provisional Application No. 61/182,611 filed May 29, 2009, entitled “Visual Displays of, and Report Generation for, Medical Data With Varying Levels of Detail,” both assigned to the Assignee of the present application and both incorporated herein in their entireties.

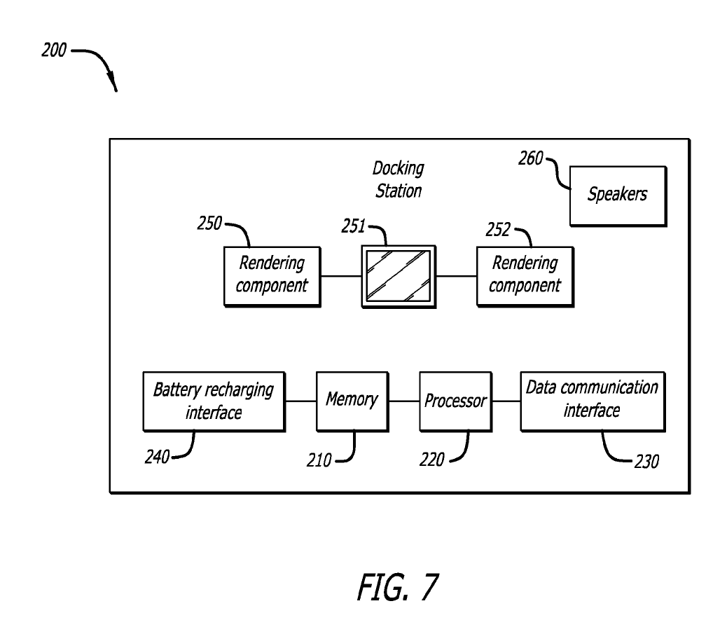

With reference to FIG. 7, a cradle 200 is illustrated in further detail. As discussed herein, the cradle is typically configured as a cradle for interlocking with an analyte monitoring device to provide operative coupling of the devices.

The cradle 200 allows for interlocking with an analyte monitoring device and includes one or more memory storage devices 210 having computer-readable code embodied thereon, the computer-readable code for retrieving data from the analyte monitoring device and uploading the retrieved data to, for example, a remote server. Thus, in an exemplary embodiment, the cradle 200 includes a processor 220; a memory 210; and a data communication interface 230 for operatively coupling to a data communication interface of an analyte monitoring device for transmission of data therefrom to the cradle 200 and uploading to a host server via the data communication interface 230 or an additional data communication interface.

The memory 210 includes stored computer-readable instructions which, when executed, causes the processor 220 to detect a connection, between the analyte monitoring device and the cradle 200, identify the connected analyte monitoring device, transfer data associated with the analyte monitoring device contained therein in a report format to cradle 200, connect with a remote server, and upload the data to the server. The memory 210 may further include a printer driver, a printer management program, or a combination thereof stored thereon. Additionally, the memory 210 may further include computer-readable instructions for automatically uploading the data to the remote server and directing a printer connected thereto to print the data.

Again with reference to FIG. 7, in various embodiments, cradle 200 may further include additional features and functionality that provide additional benefit to the user or the HCP. For example, the cradle 200 may include battery recharging interface 240 for recharging a battery of a connected device, such as an analyte monitoring device.

The cradle 200 may also include a display, such as a GUI rendering component 250 for providing an interface, such as GUI 251, that allows for user-interaction related to components displayed on the GUI 251 and for populating the GUI 251 based upon information received from a client component, a remote host server, an analyte monitoring device, a printer, or other device. The GUI rendering component 252 may provide the GUI 251 with user-controllable features to allow the user to view, enter, upload, download, or otherwise manipulate and access data and information.

The display component GUI 251 may display textual, graphical or symbolic displays which provide the user or the HCP with various types of information, such as medical and educational information, or information relating to the status of a connected device or remote server, or status of data transfer between such devices. For example, the GUI 251 may provide information confirming connection to the remote server, failure of such a connection, uploading of the data, failure of such uploading, battery status of a connected device, charging of the battery and failure of such charging.

One can also print data from the cradle 200 uploaded from a monitoring device. For example, the cradle 200 may be operatively coupled with a printer for printing of uploaded data, or the data may be further uploaded to a remote server and be printed on a printer connected to the remote serve via instructions sent from the cradle 200, either automatically, or upon a user request via the GUI 250.

Again with reference to FIG. 7, in various embodiments, the cradle 200 may further include one or more audio speakers 260 for providing the user with HCP audio messages, alarms or alerts relating to various kinds of information, such as medical information, the status of system components or data transfer. For example, the audio speaker 260 may provide audible information generated by execution of computer-readable instructions stored in the processor 220 or on the memory 210 of the cradle 200. Audible information may include conformation of a connection to a monitoring device, a remote server, failure of such a connection, status of uploading or downloading of data, failure of such uploading or downloading, status of the battery of the monitoring device, charging of the battery of the monitoring device, failure of such charging, and/or successful printing of a report.

As discussed herein, communication links from the cradle 200 to any other device may be by any suitable communication protocol for transferring data, including one or more of an Ethernet connection, RF communication protocol, an infrared communication protocol, a Bluetooth enabled communication protocol, an 802.1 1x wireless communication protocol, an equivalent wireless communication protocol, a serial or USB connection, or the like.

Generally, when configured as a cradle 200, a connected monitoring device is interlocked with the cradle 200 to comprise the analyte monitoring system described herein, such that a direct, physical connection is made between the devices and any suitable communication protocol for transferring data may be utilized. With regard to connection between the cradle 200 and a remote server, connection may be a direct wired or wireless connection to the server using any suitable protocol. For example, connection between the cradle 200 and a remote server may include a telephone line, Ethernet, or other communication protocol such that bi-directional communication between persons using the cradle 200 and using a terminal connected to the remote server is facilitated.

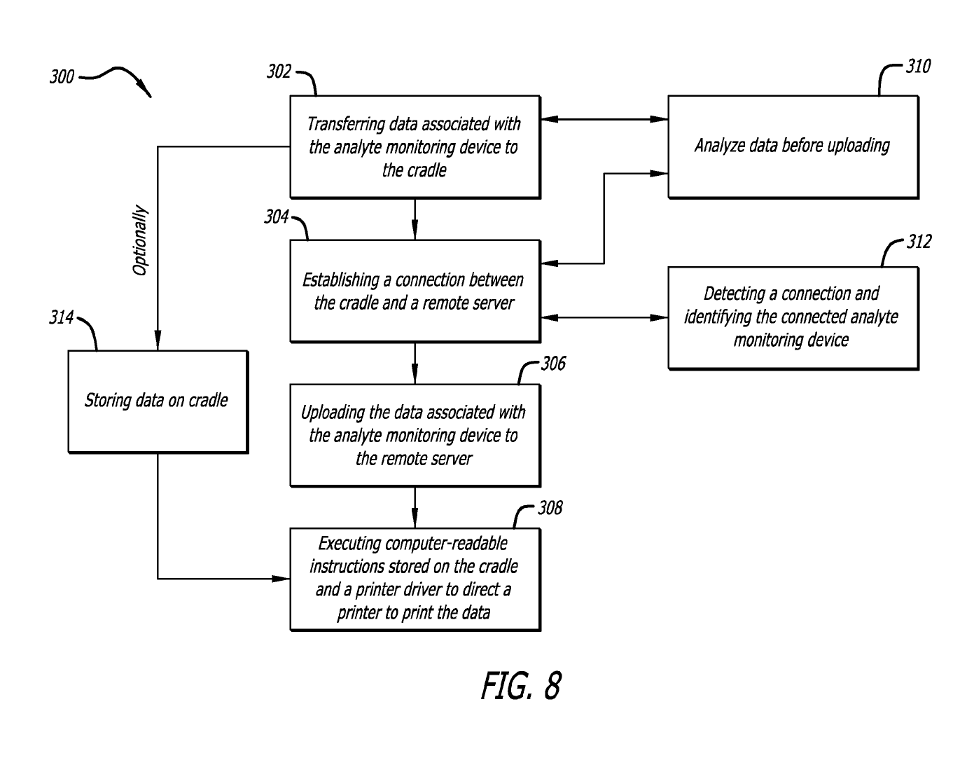

Accordingly, the invention further provides a method for establishing a connection between an analyte monitoring device and a cradle configured as a cradle for interlocking with the analyte monitoring device to facilitate data transfer to a remote server and printing of generated reports. With reference to FIG. 8, showing a flow diagram of the method, the method includes step 301 of transferring data associated with an analyte monitoring device to the intermediate cradle. A connection is established between the cradle and the remote server at 302, and data associated with the analyte monitoring device is uploaded to the remote server at 303. At 302.1, establishing the connection between the analyte monitoring device and the cradle may further include detecting a connection and identifying the connected analyte monitoring device or, at 302.2, analyzing the data (e.g., to determine if an alarm should be triggered for high or low measured analyte levels) before transfer.

The method may be performed automatically upon interlocking of the analyte monitoring device with the cradle or by prompt of a user or HCP. For example, data collected and stored on the analyte monitoring device may be automatically uploaded to a remote server upon connecting the analyte monitoring device with the cradle.

Alternatively, the method may comprise storing data on the cradle at 301.1, followed by executing computer-readable instructions present on the cradle along with a printer driver at 301.2 to direct a printer in communication with the remote server to print the data.

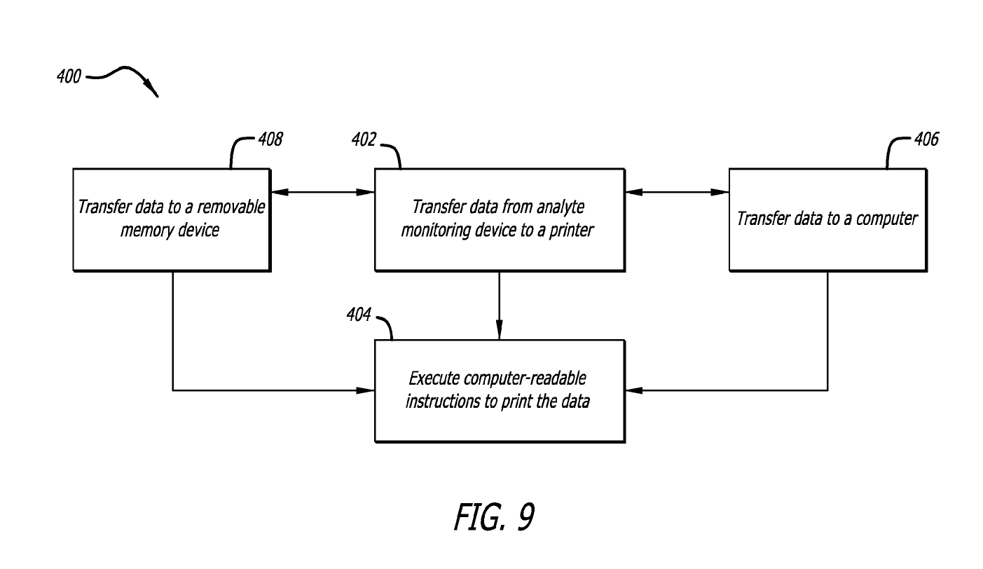

A further alternative embodiment of the invention is illustrated in FIG. 9. The analyte monitoring device may be connected directly to a printer at 401 via a wired, wireless, USB or other common data connection. The monitoring device generates a report based on data stored on the device, creates a report file in a native printer format that most printers recognize and sends this to the printer. For the preferred embodiment, the physical transport of the report to the printer is via USB and the monitor has USB host capability. Preferably, the monitor has USB OTG (on the go) capable so it can act as both a USB device which will allow it, for instance, to be charged by a USB host, or it can act as a USB host, for instance, so it can be connected directly to a printer and print a report. The generated report file format would be PDL (page description language) which is understood by most printers. The format of the data packet sent to the printer is PCP (printer control protocol), for example PJL, WPS or IEEE1284.1. When the printer receives the PDL file it will print the report. The protocol can be unidirectional from the monitor to the printer or bidirectional where the monitor can receive and act on printer status information.

Alternatively, connection to the printer can be made through a computer to which the data to be printed is transferred, at 401.1. Standard computer operating system printer capabilities can be utilized to direct printing of data from the monitoring device to a printer operably connected to the client component without need for a dedicated printer management program to be installed on the client component. To this end, the monitoring device can instead be installed with a program allowing it to mimic a removable memory device, card or drive. On its attachment to the computer, an autorun program on the monitoring device is initiated to trigger the operating system printing capabilities on the client component.

In a further alternative, connection to the printer can be made through a removable storage device to which the data to be printed is transferred, at 401.2. In this embodiment, the analyte monitoring device 150 includes a port for attachment of a removable memory device, card or drive 152 (see, FIG. 1) for download thereto of data, which removable device, card or drive 152 is inserted into a compatible client component 110 (computer or printer) for printing of data therefrom using the printing capabilities of the component's operating system or installed programs.

As used herein, “transmit” may encompass both wired and wireless forms of communication. “Memory” may encompass a single memory device or a plurality of memory devices.

It is to be understood that the invention is not limited in its application to the details of construction and the arrangement of components set forth in the description above or illustrated in the drawings. The invention is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “mounted,” “connected,” “supported,” and “coupled” and variations thereof are used broadly and encompass both direct and indirect mountings, connections, supports, and couplings, whether electrical or mechanical. Further, “connected” and “coupled” are not restricted to physical or mechanical connections or couplings.

While the system and method have been described in terms of what are presently considered to be specific embodiments, they need not be limited to the disclosed embodiments. It is intended to cover various modifications and similar arrangements included within the spirit and scope of the claims, the scope of which should be accorded the broadest interpretation so as to encompass all such modifications and similar structures. The present disclosure includes any and all embodiments of the following claims.

What is claimed is:

1. A medical method of treating a patient having diabetes mellitus through the measurement of the patient's bodily fluids and the infusion of a medication into the patient in response thereto, the method comprising:
applying blood of the patient to a test strip and inserting the test strip into a port of an insulin infusion pump;
measuring a content of a blood analyte in the blood with the insulin infusion pump;
measuring a content of an interstitial analyte with a sensor in communication with the insulin infusion pump;
consulting with a physician to analyze the blood glucose concentration and the interstitial analyte concentration and outputting, from the insulin infusion pump, information relating to the blood glucose concentration and the interstitial analyte concentration directly to a printer;
modifying a basal insulin delivery profile in the insulin infusion pump; and
administering insulin into the body of the patient in accordance with the modified basal insulin delivery profile.

2. The medical method of treatment of claim 1, wherein the blood analyte is selected from the group comprising: acetyl choline, amylase, bilirubin, cholesterol, chorionic gonadotropin, creatine kinase (e.g., CK-MB), creatine, glutamine, growth hormones, ketones, lactate, oxygen, peroxide, prostate-specific antigen, prothrombin, thyroid stimulating hormone, and troponin.

3. The medical method of treatment of claim 1, wherein the interstitial analyte is selected from the group comprising: acetyl choline, amylase, bilirubin, cholesterol, chorionic gonadotropin, creatine kinase (e.g., CK-MB), creatine, glutamine, growth hormones, ketones, lactate, oxygen, peroxide, prostate-specific antigen, prothrombin, thyroid stimulating hormone, and troponin.

4. The medical method of treatment of claim 1, wherein the blood analyte is a ketone.

5. The medical method of treatment of claim 1, wherein the interstitial analyte is a ketone.

6. The medical method of treatment of claim 1, wherein the blood analyte is lactate.

7. The medical method of treatment of claim 1, wherein the interstitial analyte is lactate.

8. The medical method of treatment of claim 1, further comprising monitoring a concentration of a drug in the body of the patient.

9. The medical method of treatment of claim 8, wherein the drug is selected from the group comprising: gentamicin, vancomycin, digitoxin, digoxin, theophylline, and warfarin.

10. The medical method of treatment of claim 1, further comprising determining a bolus insulin dose based on the measured content of the interstitial analyte.

11. The medical method of treatment of claim 10, further comprising administering the bolus insulin dose to the body of the patient with the insulin infusion pump.

12. The medical method of treatment of claim 1, further comprising calculating a chemical attribute related to the patient with the insulin infusion pump.

13. The medical method of treatment of claim 1, wherein the test strip port is an integral component of the insulin infusion pump.

14. A method of managing diabetes mellitus, comprising:
measuring a content of an analyte, wherein the analyte is selected from the group comprising: acetyl choline, amylase, bilirubin, cholesterol, chorionic gonadotropin, creatine kinase (e.g., CK-MB), creatine, glucose, glutamine, growth hormones, ketones, lactate, oxygen, peroxide, prostate-specific antigen, prothrombin, thyroid stimulating hormone, and troponin; and
outputting information relating to the analyte from a handheld device directly to a printer in a predefined report format,
whereby a course of treatment can be prescribed by a physician.

15. A medical method of treating a patient having diabetes mellitus through the measurement of the patient's bodily fluids and the infusion of a medication into the patient in response thereto, the method comprising:
measuring a content of an interstitial analyte with a sensor in communication with the insulin infusion pump;
consulting with a physician to analyze the blood glucose concentration and the interstitial analyte concentration and outputting, from the insulin infusion pump, information relating to the blood glucose concentration and the interstitial analyte concentration directly to a printer;
modifying a basal insulin delivery profile in the insulin infusion pump; and
administering insulin into the body of the patient in accordance with the modified basal insulin delivery profile,
wherein the interstitial analyte is selected from the group comprising: acetyl choline, amylase, bilirubin, cholesterol, chorionic gonadotropin, creatine kinase (e.g., CK-MB), creatine, glutamine, growth hormones, ketones, lactate, oxygen, peroxide, prostate-specific antigen, prothrombin, thyroid stimulating hormone, and troponin.

16. The medical method of treatment of claim 15, wherein the interstitial analyte is a ketone.

17. The medical method of treatment of claim 15, wherein the interstitial analyte is lactate.

18. The medical method of treatment of claim 15, further comprising determining a bolus insulin dose based on the measured content of the interstitial analyte.

19. The medical method of treatment of claim 15, further comprising monitoring a concentration of a drug in the body of the patient.

20. The medical method of treatment of claim 19, wherein the drug is selected from the group comprising: gentamicin, vancomycin, digitoxin, digoxin, theophylline, and warfarin.
